(19) United States
(12) Patent Application Publication
(10) Pub. No.: US 2002/0137698 A1
(43) Pub. Date: Sep. 26, 2002

(54) ANTISENSE INHIBITION OF RAD51

(76) Inventor: TAKANORI OHNISHI, HYOGO (JP)

Correspondence Address: FLEHR HOHBACH TEST ALBRITTON & HERBERT, FOUR EMBARCADERO CENTER, SUITE 3400, SAN FRANCISCO, CA, 941114187

(21) Appl. No.: 09/260,624

(22) Filed: Mar. 1, 1999

(51) Int. Cl. A61K 48/00; C07H 21/04

(52) U.S. Cl. 514/44; 536/23.2

(57) ABSTRACT

Described herein are methods for inhibiting cell proliferation in an individual in vivo comprising administering to the individual a composition comprising a RAD51 antisense molecule. Also described herein is a method for inhibiting the growth of a cancerous cell comprising administering to said cell a composition comprising a RAD51 antisense molecule.

### FIELD OF THE INVENTION

The invention relates to methods of inhibiting the proliferation of cells and sensitizing cells to radiation and chemotherapeutics, and in particular to treating cancerous cells and individuals in vivo (including intraoperative treatments), by antisense inhibition of the RAD51 gene.

### BACKGROUND OF THE INVENTION

The control of the proliferation of cells is of interest. For example, inhibition of the proliferation of cells is useful in treating a number of disorders such as cancer, autoimmune disease, arthritis, inflammatory bowel disease, proliferation induced after medical procedures, and many other instances. Therefore, a number of approaches have been taken which are meant to inhibit the proliferation of cells. For example, chemotherapeutics are intended to inhibit proliferation or kill cancerous cells. However, while there have been many approaches to treating disorders requiring the inhibition of cell proliferation, there is still a need to identify more efficient treatments, particularly treatments which are sensitive and which have limited side effects.

In one approach, radiation is a major treatment mode for both children and adults with high grade gliomas. Although low linear energy transfer irradiation has been shown to have some beneficial effects on the treatment of astrocytic tumors, most malignant gliomas are radioresistant so that various methods of improving the therapeutic ratio in their treatment have been explored. The efficacy of fractionated irradiation, which is commonly employed in clinical practice, depends on four facts: redistribution of tumor cells in the cell cycle, repopulation, reoxygenation, and repair of sublethal damage. These factors have generated several approaches which have been applied in clinical practice. These include accelerated fractionation so as to reduce tumor repopulation, radiosensitization of hypoxic cells by hyperbaric oxygen and nitroimidazoles, and combination with chemotherapeutic agents such as BCNU and vincristine⁽⁹⁻¹¹⁾. So far, however, none of these procedures has resulted in satisfactory outcome for the treatment of malignant gliomas.

One study has reported that RAD51 antisense inhibition enhances radiosensitivity in normal cells, in vitro. Taki, et al., Biochemical and Biophysical Res. Comm., 223:434-438 (1996). However, this study does not report on the affects of RAD51 antisense inhibition in abnormal cells, such as tumor cells, nor does this study report on the affects of RAD51 antisense inhibition in vivo.

RAD51 is of interest because it is detected in every proliferating cell. It is believed that RAD51 is within the family of proteins involved in repairing DNA damage, such as double-strand breaks in DNA caused by ionizing radiation and some alkylating agents, which lead to cell death if not repaired. Several genes related to double-strand break repair have been isolated from E. coli and S. cerevisia^{(1,2)}. In most prokaryotes, including E. coli, RecA protein or RecA-like protein plays an essential role in homologous recombination and in a variety of SOS responses to DNA damage⁽³⁾. In yeasts, which are lower eukaryotes, genes of the RAD52 epistasis group (RAD50-RAD57) have been identified by mutants not only as being deficient in their capability of DNA damage repair caused by ionizing radiation but also as having impaired capacity for mitotic and meiotic recombination^{(4,5)}. The RAD51 gene has been cloned and its product shown to be structurally similar to E. coli RecA protein with ATP-dependent DNA binding activity^{(6,7)}. One study shows a mouse homologue of the yeast RAD51 gene that functionally complements a RAD51 mutation of S. cerevisiae with sensitivity to methylmethanesulfonate, a double-strand breaking agent⁽⁸⁾.

Accordingly, it is an object of the invention to inhibit cell proliferation comprising administration of an antisense molecule that disrupts mammalian double stranded break repair. It is further an object to treat diseased cells or individuals by administering a composition comprising a RAD51 antisense molecule. It is also an object of the invention to provide methods of inhibiting RAD51 expression in vivo using antisense molecules. It is also an object of the invention to provide methods of inducing sensitization to radiation and aklylating agents in vivo using antisense molecules.

### SUMMARY OF THE INVENTION

In accordance with the objects outlined above, the present invention provides methods for inhibiting cell proliferation in an individual in vivo comprising administering to the individual a composition comprising a RAD51 antisense molecule. Also provided herein is a method for inhibiting the growth of a cancerous cell comprising administering to said cell a composition comprising a RAD51 antisense molecule.

In another aspect, provided herein is a method for inducing sensitivity to radiation and alkylating or DNA damaging chemotherapeutics in an individual in vivo comprising administering to said individual a composition comprising a RAD51 antisense molecule. Also provided herein is method for inducing sensitivity to radiation and chemotherapeutics (including alkylating agents) in a cancerous cell comprising administering to said cell a composition comprising a RAD51 antisense molecule.

In one embodiment, the methods provided herein also include the step of administering radiation or chemotherapeutic agents (including alkylating agents) to a cell.

### BRIEF DESCRIPTION OF THE DRAWINGS

FIG. 1 shows a northern blot showing the effect of antisense RAD51 oligonucleotides on RAD51 mRNA expression in glioma cells. Lane 1 shows RNA results with serum-deprived medium, lane 2 shows medium alone, lanes 3, 4, and 5 show sense, lanes 6, 7, and 8 show scrambled, and lanes 9, 10 and 11 show antisense oligonucleotides.

FIG. 2 shows a western blot showing the effect of antisense RAD51 inhibition on RAD51 protein expression in glioma cells. Lane 1 shows proteins results with serum-deprived medium, lane 2 shows medium alone, lanes 3, 4, and 5 show sense, lanes 6, 7, and 8 show scrambled, and lanes 9, 10 and 11 show antisense oligonucleotides.

FIG. 3 shows a graph indicating absorbance, wherein low numbers indicate cell death, versus radiation dose, showing the effect of antisense oligonucleotides on in vitro radiosensitivity of glioma cells. The solid triangles represent the cells which were incubated with antisense, solid circles=sense, non-solid triangles=scrambled oligonucleotides, and non-solid circle=without oligonucleotides (control).

FIG. 4 shows survival curves (survival rate versus time) for glioma-bearing mice that were treated with RAD51 oligonucleotides alone (antisense=solid line, scrambled=dashed line, and control=dotted line).

FIG. 5 shows survival curves (survival rate versus time) for glioma-bearing mice that were treated with RAD51 oligonucleotides (antisense=solid line, scrambled=dashed line, sense=dotted/dashed and control=dotted line) followed by radiation to their whole bodies.

### DETAILED DESCRIPTION OF THE INVENTION

The present invention is based on the series of discoveries relating to the pivotal role that RAD1 plays in a number of cellular functions, including those involved in disease states. In particular, the present invention is based in part on inhibiting the expression of the RAD51 gene within cells by the use of RAD51 antisense molecules. For the first time, presented herein are in vivo results which show the application of RAD51 antisense molecules. The results show the inhibition of cell growth, the inhibition of RAD51 mRNA expression, the inhibition of RAD51 protein production and the sensitization to radiation.

A RAD51 antisense molecule as defined herein is a nucleic acid molecule which inhibits expression or translation of a RAD51 nucleic acid by at least 30%, more preferably 40%, more preferably 50%, more preferably 70%, more preferably 90%, and most preferably by at least 95%. In one embodiment herein, a RAD51 antisense molecule inhibits expression or translation of a RAD51 nucleic acid by 100%.

Generally, the RAD51 antisense molecule is at least about 10 nucleotides in length, more preferably at least 12, and most preferably at least 15 nucleotides in length. The skilled artisan understands that the length can extend from 10 nucleotides or more to any length which still allows binding to the RAD51 nucleic acid. In a preferred embodiment herein, the length is about 100 nucleotides long, more preferably about 50 nucleotides, more preferably about 25 nucleotides, and most preferably about 12 to 25 nucleotides in length.

The nucleic acids herein are recombinant nucleic acids. By “recombinant” herein is meant a protein made using recombinant techniques, i.e. through the expression of a recombinant nucleic acid. A recombinant nucleic acid is distinguished from naturally occurring nucleic acid by at least one or more characteristics. For example, the nucleic acid may be isolated or purified away from some or all of the nucleic acids and compounds with which it is normally associated in its wild type host, and thus may be substantially pure. For example, an isolated nucleic acid is unaccompanied by at least some of the material with which it is normally associated in its natural state, preferably constituting at least about 0.5%, more preferably at least about 5% by weight of the total nucleic acid in a given sample. A substantially pure nucleic acid comprises at least about 75% by weight of the total nucleic acid, with at least about 80% being preferred, and at least about 90% being particularly preferred. Alternatively, the recombinant molecule could be made synthetically, i.e., by a polymerase chain reaction, and does not need to have been expressed to be formed. The definition includes the production of a nucleic acid from one organism in a different organism or host cell.

The antisense molecules are either DNA or RNA and hybridize under normal intracellular conditions to the target nucleic acid to inhibit RAD51 expression or translation. The target nucleic acid is either DNA or RNA. In one embodiment, the antisense molecules bind to regulatory sequences for RAD51. In one embodiment, the antisense molecules bind to 5′ or 3′ untranslated regions directly adjacent to the coding region. Preferably, the antisense molecules bind to the nucleic acid within 1000 nucleotides of the coding region, either upstream from the start or downstream from the stop codon. In a preferred embodiment, the antisense molecules bind within the coding region of the RAD51 molecule. In a particularly preferred embodiment, the antisense molecules have the sequences of SEQ ID NO:1 or SEQ ID NO:2.

In one embodiment, RAD51 includes homologues of RAD51. In one aspect, RAD51 homologues can be defined by the RAD51 role in recombinational repair. In another aspect, RAD51 genes encode proteins which share significant sequence identity (i.e. about 80% or greater) with residues 33-240 of E.coli RecA protein, which has been identified as a homologous core region in the literature. RAD51 homologues include RecA and RAD51 homologues in yeast and in mammals. RecA and yeast RAD51 have been cloned and are known in the art. Radding, J. Biol. Chem. 266:5355-5358 (1991); Kowalczykoswski, et al., Annu. Rev. Biochem., 63:991-1043 (1994); Basile, et al., Mol. Cell. Biol., 12:3235-3246 (1992); Aboussekhara, et al., Mol. Cell. Biol., 12:3224-3234 (1992). Genes homologous to E. Coli recA and yeast RAD51 have been isolated from all groups of eukaryotes, including mammals. Morita, et al., PNAS USA, 90:6577-6580 (1993); Shinohara, et al., Nature Genet., 4:239-243 (1993); Heyer, Experentia, 50:223-233 (1994); Maeshima, et al., Gene, 160:195-200 (1995). RAD51 has been identified in humans, mice, chicken, S. Cerevisiae, S. Pombe and Mei3 of Neurospora crassa. Human RAD51 homologues include RAD51, RAD51B, RAD51C, RAD51D, XRCC2 and XRCC3. Albala, et al., Genomics, 46:476-479 (1997); Dosanjh, et al., Nucleic Acids Res, 26:1179(1998); Pittman, et al., Genomics, 49:103-11 (1998); Cartwright, et al., Nucleic acids Res, 26:3084-3089 (1998); Liu, et al., Mol Cell, 1:783-793 (1998).

In an embodiment provided herein, the invention provides methods of treating disease states requiring inhibition of cellular proliferation. In a preferred embodiment, the disease state requires inhibition of RAD51 expression or translation. As will be appreciated by those in the art, a disease state means either that an individual has the disease, or is at risk to develop the disease.

Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. Thus, in one embodiment, the invention herein includes application to cells or individuals afflicted or impending affliction with any one of these disorders.

The compositions and methods provided herein are particularly deemed useful for the treatment of cancer including solid tumors such as skin, breast, brain, cervical carcinomas, testicular carcinomas, etc. More particularly, cancers that may be treated by the compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

The individual, or patient, is generally a human subject, although as will be appreciated by those in the art, the patient may be animal as well. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient. In a preferred embodiment, the individual requires inhibition of cell proliferation. More preferably, the individual has cancer or a hyperproliferative cell condition.

The compositions provided herein may be administered in a physiologically acceptable carrier to a host, as previously described. Preferred methods of administration include systemic or direct administration to a tumor cavity or cerebrospinal fluid (CSF). The route of administration is in accord with known methods, e.g. injection or infusion by intravenous, intraperitoneal, intracerebral, intramuscular, intraocular, intraarterial or intralesional routes, topical administration, or by sustained release systems.

One can use methods of injection or directly apply the compositions during surgery. The concentration of therapeutically active compound in the formulation may vary from about 0.1-100 wt. %.

In a preferred embodiment, these compositions can be administered to a cell or patient, as is outlined above and generally known in the art for gene therapy applications. In gene therapy applications, the antisense molecules are introduced into cells in order to achieve inhibition of RAD51. “Gene therapy” includes both conventional gene therapy where a lasting effect is achieved by a single treatment, and the administration of gene therapeutic agents, which involves the one time or repeated administration of a therapeutically effective DNA or RNA. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]).

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The antisense molecules can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

In addition to inhibition RAD51 expression or translation, the antisense molecules herein induce sensitivity to alkylating agents and radiation. Induced sensitivity (also called sensitization or hypersensitivity) can be measured by the cells tolerance to radiation or alkylating agents. For example, sensitivity, which can be measured, i.e., by toxicity, occurrs if it is increased by at least 20%, more preferably at least 40%, more preferably at least 60%, more preferably at least 80%, and most preferably by 100% to 200% or more.

In an embodiment herein, the methods comprising administering the antisense molecules provided herein further comprise administering an alkylating agent or radiation. For the purposes of the present application the term ionizing radiation shall mean all forms of radiation, including but not limited to alpha, beta and gamma radiation and ultra violet light, which are capable of directly or indirectly damaging the genetic material of a cell or virus. The term irradiation shall mean the exposure of a sample of interest to ionizing radiation, and term radiosensitive shall refer to cells or individuals which display unusual adverse consequences after receiving moderate, or medically acceptable (i.e., nonlethal diagnostic or therapeutic doses), exposure to ionizing irradiation. Alkylating agents include BCNU, CCNU, MMS, and cross-linkers including cis-Pt and carbo-PT.

In one embodiment herein, the antisense molecules provided herein are administered to prolong the survival time of an individual suffering from a disease state requiring the inhibition of the proliferation of cells. In a preferred embodiment, the individual is further administered radiation or an alkylating agent.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are specifically incorporated by reference.

### EXAMPLES

### Example 1

### Materials and Methods

Cells and cell culture. An established mouse glioma cell line, 203G, an ependymoblastoma derived from C57BL67 mouse, was used for the present study. It is a transplantable tumor of cerebral origin and the growth pattern and the responsiveness to chemotherapeutic drugs and irradiation are known to be consistent with human malignant gliomas⁽¹³⁾. The glioma cells were maintained in DMEM containing 10% FBS at 37° C. under standard conditions of 100% humidity, 95% air and 5% CO₂.

Synthetic oligodeoxynucleotides. Two antisense phosphorothioates for RAD51 oligonucleotides (antisense 1:GGCTT-CACTAATTCC (SEQ ID NO:1) [368-382], antisense 2:CGTATGACAGA-TCTG (SEQ ID NO:2) [578-592], two sense and two scrambled control phosphorothioate oligonucleotides, sense 1:GGAATTAGTGAAGCC (SEQ ID NO:3), sense 2:CAGATCTGTCATACG (SEQ ID NO:4); scrambled 1:TCGCGA-TCACCTTAT (SEQ ID NO:5), scrambled 2:ACGGTACGCTTAAGT (SEQ ID NO:6)) were synthesized with an Applied Biosystem 394 DNA, RNA Synthesizer (Perkin-Elmer Corporation, CA). These oligonucleotides were deprotected on the column, dried, resuspended in Tris-EDTA (10 mM Tris, pH 7.4 and 1 mM EDTA, pH 8.0), and quantified by means of spectrophotometry.

Treatment of cells with synthetic oligonucleotides. The exponentially grown glioma cells were washed twice with prewarmed DMEM and incubated with 5 ml of DMEM containing a mixture of IDNs and Lipofectamine reagent (Gibco BRL, MD). After 5 hours incubation at 37° C. under standard conditions, an equal volume of DMEM containing 20% FBS was added to the cells. Seven hours later, the cells were again incubated with the same mixture of oligonucleotides and Lipofectamine for 5 h, and this procedure was repeated after an interval of 12 hours.

RNA assay. Cells treated with the synthetic oligonucleotides were lysed in a solution of guanidine isothiocyanate, after which RNA was extracted by acid phenol and isopropanol precipitation. The RNAs were separated on 1% MOPS-formaldehyde gel and transferred to Hybond-N-(Amersham Int. plc, Buckinghamshire, UK). The filters were hybridized either with a 1.9 kb BamHI-EcoRV fragment derived from mouse Rad51 cDNA, or with a 1.1 kb fragment of human GAPDH cDNA. The filters were washed in 0.2×SSC/0.1% SDS at 55° C.

Western blotting. At the end of incubation with the oligonucleotides, the cells were washed with DMEM and harvested by trypsinization. The cells were then lysed with an 8% SDS solution, and diluted with PBS solution containing 5% 2-mercaptoethanol, 10% glycerol and 0.5% bromophenol blue for final SDS concentration of 2%. After the protein solutions were separated by 7.5% SDS polyacrylamide gel electrophoresis, they were electrophoretically transferred onto an Immobilon-P membrane (Millipore, Bedford, Mass.). The membrane was incubated with a primary antibody against RAD51 protein⁽¹⁴⁾ (1:1000) at 4° C. overnight. After being washed in 0.1M Tris-HCl buffered saline with 0.1% Tween, 20 (TTBS), the membrane was processed with ECL Western blotting system (Amsersham). Briefly, the membrane was reacted with horseradish peroxidase-labeled secondary antibody (1:1000 dilution for horseradish peroxidase-linked anti-rabbit immunoglobulin) for 1 hours at room temperature. Rinsed membrane was then incubated in equal volumes of detection reagent 1 and detection reagent 2 for 1 min. After draining off the detection reagents, the membrane was wrapped. Hyperfilm-ECL (Amersham) was put on the membrane, exposed for 1 min. and developed.

In vitro treatment and cytotoxicity assay. Mouse 203G glioma cells were treated with 100 nM antisense, sense or scrambled oligonucleotides three times every 12 hours in the same way as described above. Immediately after the third treatment, the cells were irradiated with graded doses administered with a ¹³⁷Cs unit (1.37 Gyman: Gammacell 40 Exactor, Nordion International Inc., Canada). The irradiation dose was determined by thermoluminescence dosimetry. Six hours after irradiation, the cells were washed, harvested and then seeded in a 96-well microculture plate (Nunc, Denmark) at a concentration of 2×10⁴ cells per well. After 48 hours incubation at 37° C. under standard conditions, the cells were incubated with a medium containing 5 mg/ml of MTT (Sigma, St. Louis, Mo.). Four hours later, an equal volume of 10% SDS in 0.01 NDCl was added to the medium. After incubation at 37 overnight, the absorbance of the wells was measured on a microplate reader (MTP-120, Corona, Tokyo, Japan) with a test radiation dose resulting in 50% inhibition of MTT dye formation, compared to controls with no radiation (ID₅₀), was estimated by plotting the percentage of control OD₅₇₉ against the radiation dose (Gy).

In vivo treatment study. Exponentially grown mouse 203G glioma cells were harvested and suspended in DMEM without serum at a concentration of 5×10⁷ cells/ml. A volume of 100 ml of the cell suspension was percutaneously inoculated into the cisterna magna of male C57BL/6J mice (6 weeks old, SLC, Hamaroatsu, Japan) by using a 27 gauge needle under mild anesthesia with dimethyl ether. The accuracy of the inoculation was confirmed by the oposhotonic response of the mice. Twelve hours later, 50 ml of DMEM containing a mixture of 2 mM each of oligonucleotide and Lipofectamine was administered to the mice via the cisterna magna. This administration of oligonucleotides was carried out two more times every 12 hours. For the radiation treatment group, entire bodies of the mice were irradiated with a dose of 6 Gy soon after the third administration of oligonucleotides. Irradiation consisted of 180 kVp X-rays at a dose rate of 0.76 Gy/min.

### Results

Effects of Antisense Oligonucleotides on RAD51 mRNA and Protein Expression

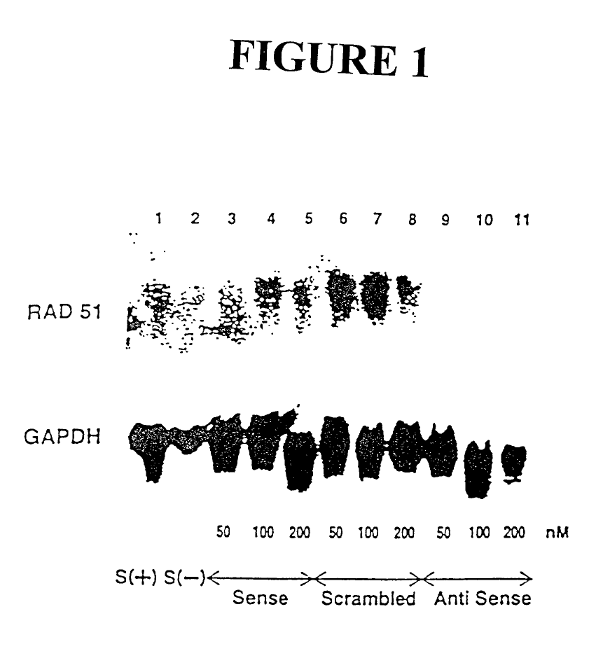

Effect of antisense RAD51 oligonucleotides on RAD51 mRNA expression are shown in FIG. 1. FIG. 1 shows RNA was extracted from 203G glioma cells after incubation with serum-deprived medium (lane 1) or medium alone (lane 2), sense (lanes 3, 4, 5), scrambled (lanes 6, 7, 8) or antisense oligonucleotides (lanes 9, 10, 11) for 29 hours at the indicated concentrations. RNAs were fractionated on an agarose gel and RAD51 expression was determined by Northern blotting analysis. Equal loading of total RNA was ensured prior to spectrophotometric quantification of RNA and subsequent reprobing GAPDH cDNA.

The two 15-bp antisense oligonucleotides were complementary to a region showing homology between mouse and human sequences. RAD51 antisense oligonucleotides significantly reduced the level of RAD51 mRNA compared to the effect of sense and scrambled oligonucleotides (FIG. 1). Scanning densitometry showed that the RAD51 mRNA level was reduced by about 90% and by more than 95% after three courses of the treatments with 50 nM and 100 nM of antisense oligonucleotides, respectively, and that no detectable mRNA was produced in the cells treated with 200 nM of antisense oligonucleotides. The effect of both scrambled and sense oligonucleotides on the expression of RAD51 mRNA was minor with the concentrations used in this study, but serum-deprived medium reduced the mRNA level by 40% compared to the control (FIG. 1).

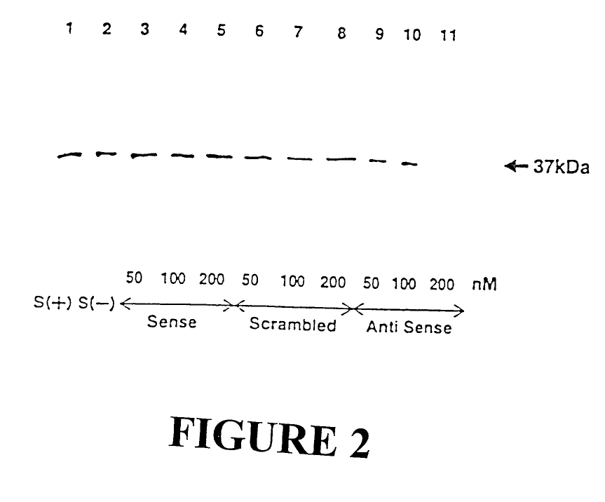

FIG. 2 shows the effect of antisense RAD51 inhibition on RAD51 protein expression in mouse 203G glioma cells. The extent of the protein expression was visualized with the enhanced chemiluminescence method. The level of RAD51 protein with a molecular weight of 37 kDa is reduced by antisense inhibition in a dose-dependent manner. Western blot showed that 100 nM and 200 nM of antisense oligonucleotides reduced the expression of RAD51 protein by 70% and 90%, respectively. Neither sense nor scrambled RAD51 oligonucleotides had such an inhibitory effect on RAD51 protein expression (FIG. 2).

Effects of Antisense Oligonucleotides on in Vitro Radiosensitivity of Glioma Cells

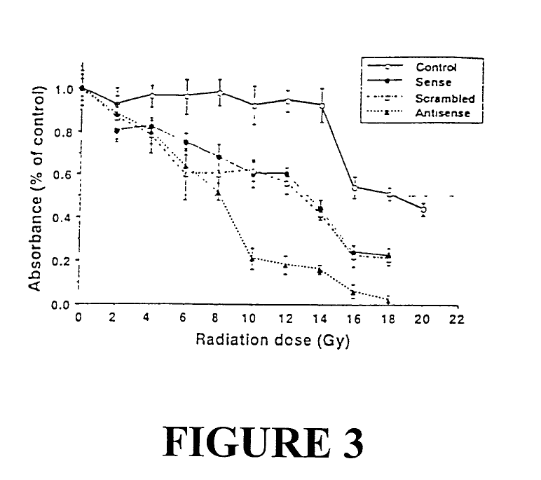

FIG. 3 shows the effect of antisense oligonucleotides on in vitro radiosensitivity of glioma cells. Cytotoxicity was evaluated with MTT assay. The cells were incubated with antisense, sense or scrambled oligonucleotides, or without oligonucleotides (control) for 29 hours followed by irradiation with the indicated doses. Data are expressed as means SD for separate four experiments. ID₅₀ was determined as a radiation dose resulting in 50% inhibition of controls with no irradiation.

Treatment of 203G glioma cells in culture with 100 nM of antisense oligonucleotides significantly potentiated their radiation sensitivity compared to radiation treatment alone (ID₅₀:8.41.1 vs 18.51.1. p<0.001. paired t-test) and treatment with sense or scrambled oligonucleotides (ID₅₀:12.80.8 (sense) and 12.51.0 (scrambled), p<0.005 and p<0.01, respectively, paired t-test) (FIG. 3). Both sense and scrambled oligonucleotides also enhanced the radiosensitivity of the cells at radiation doses of less than 8 Gy, but at doses of more than 10 Gy, their effect was significantly less than that of antisense oligonucleotides. The two different types of antisense oligonucleotides showed almost the same radio-enhancing effect on the glioma cells.

Effects of Antisense Oligonucleotides on in Vivo Radiosensitivity in Tumor-Bearing Mice

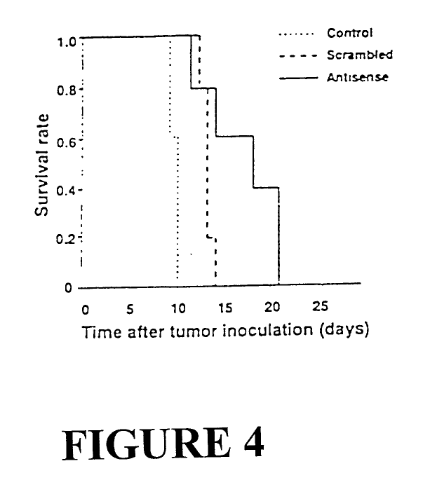

FIG. 4 shows the survival curves for glioma-bearing mice that were treated with RAD51 oligonucleotides (antisense or scrambled) along. The number of mice is 10 for each different type of treatment. Antisense vs. control: (mean survival time) 17.0 vs. 9.6 days (p<0.001); Antisense vs. scrambled: 17.9 vs. 13.0 (p<0.01) (Logrank test). When 203G glioma-bearing mice were treated with RAD51 antisense oligonucleotides alone, the survival time of the mice was significantly prolonged compared to controls or mice treated with scrambled oligonucleotides (p<0.001 or p<0.01, respectively, logrank test) (FIG. 4).

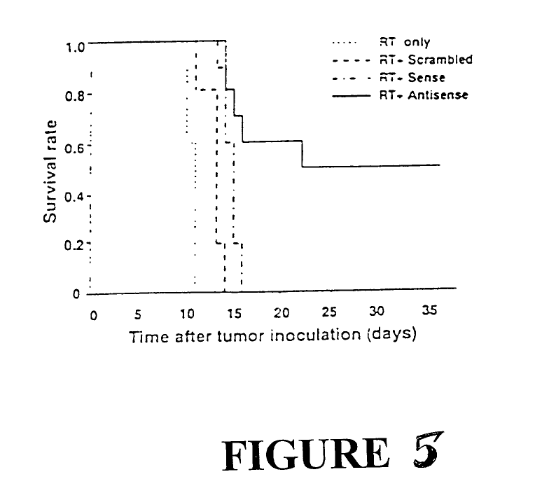

FIG. 5 shows the survival curves for glioma-bearing mice that were treated with RAD51 oligonucleotides (antisense, sense or scrambled) followed by 6 Gy radiation to their whole bodies. The number of mice is 10 for each different type of treatment. All surviving mice with antisense treatment were sacrificed at 36 days after tumor inoculation. Antisense vs. control: (mean survival time) 19.1 vs. 10.6 days (p<0.0001); Antisense vs. sense: 19.1 vs. 14.7 days (p<0.01); Antisense vs. scrambled: 19.1 vs. 12.8 days (p<0.0001) (Logrank test).

The combination of RAD51 antisense oligonucleotides and 6 Gy irradiation extended survival time much longer than did either treatment with radiation only or administration of the antisense alone (p<0.0001 and p<0.01, respectively, logrank test) (FIG. 5). In addition, the radio-enhancing effect of the antisense oligonucleotides was significantly stronger than that of the sense and scrambled oligonucleotides (p<0.01 and p<0.001, respectively; longrank test). Anatomical and histological studies of dead mice revealed that a growing tumor mass occupied the basal cistern and cisterna magna and severely compressed the brain and spinal cord. In the dead mice treated with antisense oligonucleotides, fluorescence of FITC, which was labeled for the antisense oligonucleotides, was visualized in the tumor cells but a little in the normal tissues of brain and spinal cord.

### REFERENCES

1. Roca, A. T., and Cox, M. M. (1990) Crit. Rev. Biochem. Mol. Biol. 25:415-456/

2. Shinohara, A., Ogawa, H., and Ogawa, T. (1992) Cell 69:457-470.

3. Kowalczykowski, S. C. (1991) Annu. Rev. Biophys. Biophys. Chem. 20:539-575.

4. Resnick, M. A. (1987) in Meiosis (Moens. P. B., Ed.), pp 157-210. Academic Press, New York.

5. Friedberg, E. C. (1988) Microbiol. Rev. 52:70-102.

6. Aboussekhara, A., Chanet, R., Adjiri, A., and Fabre, F. (1992) Mol. Cell. Biol. 12:3224-3234/

7. Basile, G., Aker, M., and Mortimer, R. K. (1992) Mol. Cell. Biol. 12:3235-3246.

8. Morita, T., Yoshimura, Y., Yamamoto, A., Murata, K., Mori, M., Yamamoto, H., and Matsushiro, A. (1993) Proc. Natl. Acad. Sci. USA 90:6577-6580.

9. Thames, H. D., Peters, L. J., and Winthers, H. R. (1983) Int. J. Radiat. Oncol. Biol. Phys. 9:127-138.

10. Reddy, E. K., Kimler, B. F., Henderson, S. D., and Morantz, R. A. (1980) in Radiation Sensitizers: Their Use in the Clinical Management of Cancer (Brady, L. W., Ed.), pp 457-471, Masson, New York.

11. Wheeler, K. T., and Kaufman, K. (1981) Int. J. Radiat. Oncol. Biol. Phys. 7:1065-1066.

12. Taki, T., Ohnishi, T., Yamamoto, A., Hiraga, S., Arita, N., Izumoto, S., Hayakawa, T., and Morita, T. (1996) Biochem. Biophys. Res. Commun. 223:434-438.

13. Muller, P. J., Shin, K. H., and Shin, D. H. (1983) Can. J. Neurol. Sci. 10:105-109.

We claim:

1. A method for inhibiting cell proliferation in an individual in vivo comprising administering to said individual a composition comprising a RAD51 antisense molecule.

2. A method for inducing sensitivity to radiation in an individual in vivo comprising administering to said individual a composition comprising a RAD51 antisense molecule.

3. A method for inducing sensitivity to a chemotherapeutic agent in an individual in vivo comprising administering to said individual a composition comprising a RAD51 antisense molecule.

4. A method for inhibiting the growth of a cancerous cell comprising administering to said cell a composition comprising a RAD51 antisense molecule.

5. A method for inducing sensitivity to radiation in a cancerous cell a composition comprising RAD51 antisense molecule.

6. A method for inducing sensitivity to a chemotherapeutic agent in a cancerous cell a composition comprising RAD51 antisense molecule.

7. A nucleic acid molecule having a sequence selected from the group consisting of SEQ ID NO:1 and SEQ ID NO:2.

8. The method of claim 1 further comprising the step of administering radiation.

9. A method of prolonging the survival of an individual comprising administration to said individual a Rad51 antisense molecule.

10. A method of treating cancer in an individual comprising administration to said individual a Rad51 antisense molecule.

11. A method according to claim 10 wherein said administration comprises localized delivery of said Rad51 antisense molecule.

12. A method according to claim 10 wherein said administration comprises localized delivery of said Rad51 antisense molecule and said method further comprises radiation treatment of said patient.

13. A method according to claim 10 wherein said administration comprises localized delivery of said Rad51 antisense molecule and said method further comprises chemotherapeutic treatment of said patient.

14. A kit for diagnosing and/or treating cancer comprising a Rad51 antisense molecule.
